(19) United States
(12) Patent Application Publication
(10) Pub. No.: US 2012/0323016 A1
(43) Pub. Date: Dec. 20, 2012

(54) METHOD OF PREPARING DERIVATIVES/OLIGOMERS OF EPICATECHIN AND APPLICATIONS THEREOF

(75) Inventors: Dejian Huang, Singapore (SG); Wei Chen, Singapore (SG); Runyun Ni, Singapore (SG); Caili Fu, Singapore (SG); Yi Ling Quek, Singapore (SG)

(73) Assignee: NATIONAL UNIVERSITY OF SINGAPORE, Singapore (SG)

(21) Appl. No.: 13/516,744

(22) PCT Filed: Dec. 17, 2010
(86) PCT No.: PCT/IB2010/055902

§ 371 Date: Aug. 28, 2012

(30) Foreign Application Priority Data
Dec. 17, 2009 (US) 61287281

(51) Int. Cl. C07D 311/32; C07D 311/62; C07F 15/00; C07D 405/04

(52) U.S. Cl. 548/364.4; 548/525; 549/399; 549/400; 549/210

(57) ABSTRACT

The present invention relates to the preparation of a class of novel, optically active compounds derived from oligomeric proanthocyanidins (OPCs), more particularly to oligomers of epicatechin. These compounds include multidentate ligands and their metal complexes for use in catalysis. Methods of depolymerising proanthocyanidins to form catechins, and particularly depolymerising proanthocyanidins derived from plant sources to form novel epicatechins having application in catalysis are disclosed.

### FIELD OF THE INVENTION

The present invention relates to the preparation of a class of novel, optically active compounds derived from oligomeric proanthocyanidins (OPCs), more particularly to oligomers of epicatechin. These compounds include multidentate ligands and their metal complexes for use in catalysis.

### BACKGROUND ART

The following discussion of the background art is intended to facilitate an understanding of the present invention only. It should be appreciated that the discussion is not an acknowledgement or admission that any of the material referred to was part of the common general knowledge as at the priority date of the application.

Asymmetric organic reactions with high stereoselectivity have been a major research field for organic and organometallic chemists. This continues to be one of the main focus points of chemical research driven both by intellectual challenges and the ever increasing demand of pharmaceutical and agrochemical industry for entiomerically/optically pure compounds as bioactive agents. Since 2001, drugs with racemic mixtures are no longer registered by the Food and Drug Administration of the United States. There are great demands for innovations particularly in environmentally friendly and economically viable alternative to carry out asymmetric organic reactions that eventually will not only help lower the hefty price of the new drugs but also produce less or no chemical pollution by making the process “green”.

Replacing costly synthetic chiral ligands with naturally occurring and cheap ones would be a way to this goal. Mother Nature provides an unlimited source of optically pure compounds as synthetic targets, chiral resolution reagents, organocatalysts, and chiral ligands. Among them, tartaric acid, alkaloids, sugar, and amino acids have received the most attention and a number of “privileged” catalysts components have been derived from these compounds (Yoon, T. P.; Jacobsen, E. N. Privileged chiral catalysts. Science (Washington, D.C., United States) (2003), 299(5613), 1691-1693). In sharp contrast, little attention has been paid to one of the most abundant plant secondary metabolites—oligomeric proanthocyanidins. To this end, oligomeric proanthocyanidins (OPCs) may have enormous potential waiting to be explored.

Structurally, OPCs have some similarity with (R or S)-BINAP, a “privileged” chiral ligand found many application in asymmetric organic reactions (Berthod, M. I.; Mignani, G.; Woodward, G.; Lemaire, M. Chem. Rev. 2005, 105, 1801 1836). However, BINAP is optically active and involves a number of process steps in synthesis, whereas, OPCs are readily available from biomass. Abundantly present in agricultural products and forestry wastes such as pine barks, mangosteen peels, cocoa bean, grape seeds, and sorghum bran, OPCs are well known as potent antioxidant supplements that may have health benefits on delaying the onset of chronic diseases.

The figure below illustrates typical structures of OPCs with epicatechin as the monomeric unit. Oligomer A is the A-type 4-8 linkage most commonly seen in nature (n=2-50). Oligomer B is the B-type 4-6 linkage, and oligomer C is the A-type 4,8 linkage. B-type and A type linkages can co-exist in one oligomer chain.

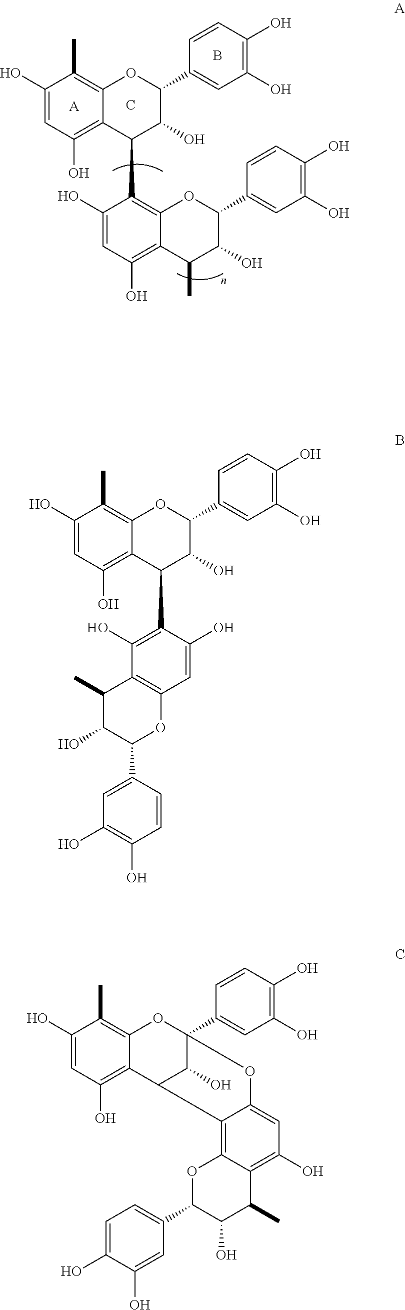

Previous arts have demonstrated that OPCs can be depolymerized by nucleophiles in the presence of acid. A range of depolymerized products were reported this way with different types of nucleophiles such as mercaptotoluene, alkyl thiols, cysteine and its derivatives, etc. The utility of the products has been documented to a certain extent, particularly for their therapeutic effectiveness (Torres, J. L.; Lozano, C.; Julia, L.; Sanchez-Baeza, F. J.; Anglada, J. M.; Centelles, J. J.; Cascante, M. Cysteinyl-flavan-3-ol Conjugates from Grape Procyanidins. Antioxidant and Antiproliferative Properties. Bioorganic & Medicinal Chemistry (2002), 10(8), 2497-2509. Torres, J. L.; Lozano, C.; Maher, P. Conjugation of catechins with cysteine generates antioxidant compounds with enhanced neuroprotective activity. Phytochemistry (Elsevier) (2005), 66(17), 2032-2037.).

Throughout the specification unless the context requires otherwise, the word “comprise” or variations such as “comprises” or “comprising”, will be understood to imply the inclusion of a stated integer or group of integers but not the exclusion of any other integer or group of integers.

Throughout the specification unless the context requires otherwise, the word “include” or variations such as “includes” or “including”, will be understood to imply the inclusion of a stated integer or group of integers but not the exclusion of any other integer or group of integers.

Throughout the specification unless the context requires otherwise, the term “Ar” and “Ar′”, will be understood to refer to the same substituted functional groups.

### SUMMARY OF THE INVENTION

While the utility of the depolymerized products of OPC has been documented to a certain extent, particularly for their therapeutic effectiveness, no previous art has reported on the application of using such compounds as asymmetric catalysts. This invention resides in the synthesis of novel chiral multidentate ligands and their transition metal complexes for use in asymmetric catalysis.

Oligomeric proanthocyanidins (OPCs) are major secondary metabolites found abundantly in plant kingdom, including those of agricultural byproducts like mangosteen pericarps, peanut skins, and grape seeds etc. OPC typically compose of repeating units of epicatechin or catechin. The hydroxyl groups of two monomer units are positioned ideally for chelating transition metals forming chiral complexes. With simple protection of the ortho-dihydroxyl groups on the B ring from competitive binding of metals, a new chiral ligand is obtained which can be complexed with a metal and used with effect in catalyzing organic reactions.

In accordance with one aspect of the invention there is provided a method for modifying a compound having repeat units of

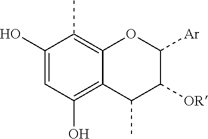

wherein Ar (≡Ar′) represents a substituted functional group selected from a group consisting of: a phenyl, a hydroxyphenyl, a dihydroxyphenyl, an alkoxy, an ester, an alkyl group, and a alkoxyphenyl group; the method comprising depolymerizing the compound with a nucleophile in the presence of acid.

The nucleophile may be selected from compounds containing sulphur, carbon, nitrogen, iodine, phosphorus, or arsenic.

The carbon nucleophile may be selected from heterocyclic compounds, aromatic compounds, acyclic organic compounds or small inorganic anions.

The heterocyclic compounds may include pyrroles, pyrazoles, indoles, furan, benenzofuran, thiophene, benzothiophene and any combination thereof. The aromatic compounds may include phenols, anilines, naphthol and naphthylamines and any combination thereof. The acyclic organic compounds may include olefins, alkynes, acetonylacetonate, acetylacetate, and their derivatives, vinyl ethers, and vinyl amines and any combination thereof. The small inorganic anions may include sulfite, thiosulfite, cyanide, thiocyanide, iodide, hydrogen sulfide, phosphide and any combination thereof.

The hydroxylphenyl group may include 2-hydroxyphenyl, 3-hydroxyphenyl, 4-hydroxyphenyl, 3,4-dihydroxyphenyl, 3,5-dihydroxyphenyl, 4,6-dihydroxyphenyl, 3,4,5-trihydroxyphenyl, 4-hydroxy-3-methoxyphenyl.

In accordance with a preferred feature of the invention the method may further comprise selectively protecting the hydroxyl groups in the repeat unit

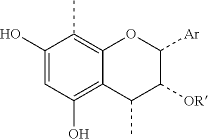

by adding a mixture of the compound having the repeat units in a polar aprotic solvent to dimethylaminopyridine and methyl propiolate.

The polar aprotic solvent may include one selected from a group consisting of: dimethyl sulfoxide (DMSO), acetone, methylethyl ketone, acetonitrile, tetrahydrofuran, N,N-dimethylformamide.

In accordance with a second aspect of the invention there is provided a method of synthesising a catechin from an oligomeric proanthocyanidin, comprising modifying selected polar oxygen containing groups with an unsaturated hydrocarbon or hydrocarbon derivative compound to prevent competitive binding of metals thereto, to form a modified oligomeric proanthocyanidin, and depolymerising the modified oligomeric proanthocyanidin to form said catechin in the form of a chiral ligand.

The method of the invention has particular application in synthesising a catechin from an oligomeric proanthocyanidin. The intermediate oligomer is a modified oligomeric proanthocyanidin.

The unsaturated hydrocarbon compound may be an alkyne or alkyne derivative.

In one highly preferred embodiment, the unsaturated hydrocarbon compound is a terminal alkyne or terminal alkyne derivative.

According to a specific example, the modification of the oligomeric proanthocyanidin is by reaction with propynoate methyl ester (methyl propiolate).

According to a more specific example, the modification of the oligomeric proanthocyanidin is by reaction in a polar aprotic solvent with propynoate methyl ester and N,N-dimethylpyridine.

The polar aprotic solvent may be selected from the group consisting of: dimethyl sulfoxide (DMSO), acetone, methylethyl ketone, acetonitrile, tetrahydrofuran, N,N-dimethylformamide.

The oligomeric proanthocyanidin may conveniently have epicatechin as the monomeric unit.

It is most preferred that the selected polar oxygen containing groups comprise at least one hydroxyl group on the B ring of the epicatechin.

In the step of depolymerizing, the modified oligomeric proanthocyanidin may be depolymerized with a nucleophile in the presence of an acid.

The nucleophile may be selected from compounds containing iodine, phosphorus, sulphur, nitrogen, carbon, or arsenic.

Where the nucleophile is a carbon nucleophile, it may be selected from heterocyclic compounds, aromatic compounds, acyclic organic compounds or small inorganic anions.

The heterocyclic compounds may include pyrroles, pyrazoles, indoles, furan, benenzofuran, thiophene, benzothiophene and any combination thereof. The aromatic compounds may include phenols, anilines, naphthol and naphthylamines and any combination thereof. The acyclic organic compounds may include olefins, alkynes, acetonylacetonate. acetylacetate, and their derivatives, vinyl ethers, and vinyl amines and any combination thereof. The small inorganic anions may include sulfite, thiosulfite, cyanide, thiocyanide, iodide, hydrogen sulfide, phosphide and any combination thereof.

The hydroxylphenyl group may include 2-hydroxyphenyl, 3-hydroxyphenyl, 4-hydroxyphenyl, 3,4-dihydroxyphenyl, 3,5-dihydroxyphenyl, 4,6-dihydroxyphenyl, 3,4,5-trihydroxyphenyl, 4-hydroxy-3-methoxyphenyl.

In accordance with a third aspect of the invention there is provided a catechin metal complex comprising a catechin formed according to the above method complexed with a metal. The catechin may be epicatechin.

The metal may advantageously be selected from one or more of an alkali metal, and alkali earth metal, a transition metal, a lanthanide or an actinide.

In accordance with a fourth aspect of the present invention there is provided a compound having at least one unit of a general formula:

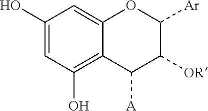

wherein Ar represents a substituted functional group selected from a group consisting of: a hydroxyphenyl, a dihydroxyphenyl, an alkoxyphenyl, ester, alkyl, alkoxyphenyl; and wherein A represents a substituted functional group.

A may include a nucleophile containing iodine, phosphorus, sulphur, oxygen, nitrogen, hydrogen, carbon, and any combination thereof.

Where the nucleophile A contains carbon, the nucleophile may be selected from the group consisting of: carbon-carbon single bonds, carbon-carbon double bonds, carbon-carbon triple bonds, nitrogen-carbon single bonds, nitrogen-carbon double bonds, sulphur-carbon single bond, oxygen-carbon single bond oxygen-carbon double bond, carbon-phosphine single bond, carbon iodine single bond, and any combination thereof.

The compound may further include one, two, or three metals selected from the group consisting of: alkali metals, alkali earth metals, transition metals, lanthanides, actinides, and metalloids.

The compound may further include a ligand (or donor atom) bound to the metal.

The ligand may be a monodentate, bidentate, tridentate, tetradentate and pentadentate ligand. The donor atom can include oxygen, nitrogen, sulphur, phosphorus, and carbon.

In accordance with a fifth aspect of the present invention there is provided a compound having at least one unit of a general formula:

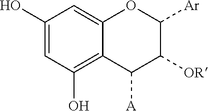

wherein Ar represents a substituted functional group selected from a group consisting of: a hydroxyphenyl, a dihydroxyphenyl, an alkoxyphenyl, phenol, ester, alkyl, alkoxyphenyl; where R′ is selected from hydrogen, any carbon containing moiety or other functional group; and wherein A is selected from one of:
any moiety or moieties containing iodine or phosphorus;
a group having the formula —SCH₂CR_{A}R_{B}, where R_{A} is any functional group or moieties containing functional groups, and R_{B} is any group containing sulphur, or nitrogen and/or a cyclic, heterocyclic, polycyclic or polyheterocyclic moiety;
a group having the formula —S—CH₂CH—YR_{B}, where R_{B} is any group, and Y is sulphur, or nitrogen as a secondary or tertiary amine or an imine;
a group having the formula —S—R_{C}—YR_{B}, where Y is sulphur, or nitrogen as a secondary or tertiary amine or an imine, R_{B} is any group, and R_{C} is any group;
a group having the formula —R_{C}—YR_{B}, where Y is sulphur, or nitrogen as a secondary or tertiary amine or an imine, R_{B} is any group, and R_{C} is any group
a group selected from:

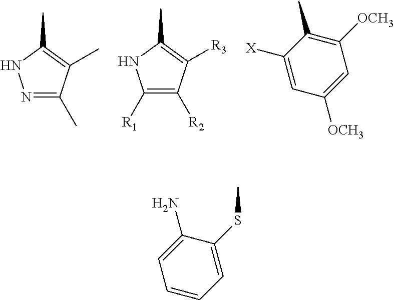

where X is selected from —OH and —NH₂, and R₁, R₂, R₃ are any group.

The group identified as Ar may comprise:

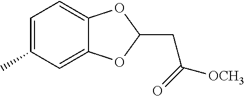

The compound may, in a particularly advantageous embodiment, have the general formula:

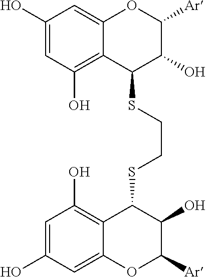

The compound may further form a metal complex with a metal selected from the group consisting of: alkali metals, alkali earth metals, transition metals, lanthanides, actinides, and metalloids. Preferably the complex is a coordination complex bonding with at least near oxygen atoms of hydroxyl groups.

The compound may further include a ligand (or donor atom) bound to the metal.

The ligand may be a monodentate, bidentate, tridentate, tetradentate and pentadentate ligand. The donor atom can include oxygen, nitrogen, sulphur, phosphorus, and carbon.

In accordance with a sixth aspect of the present invention there is provided a compound having first and second units of a general formula:

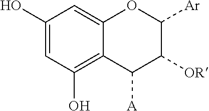

wherein Ar represents a substituted functional group selected from a group consisting of: a hydroxyphenyl, a dihydroxyphenyl, an alkoxyphenyl, phenol, ester, alkyl, alkoxyphenyl; where R′ is selected from hydrogen, any carbon containing moiety or other functional group; and
wherein A is selected from any moiety or moieties containing iodine, phosphorus, sulphur, arsenic, carbon, nitrogen or oxygen and said first unit and said second unit are connected by A.

The group identified as Ar may comprise:

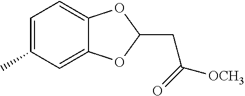

The compound may, in a particularly advantageous embodiment, have the general formula:

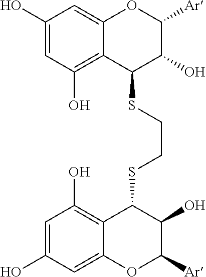

The compound may further form a metal complex with a metal selected from the group consisting of: alkali metals, alkali earth metals, transition metals, lanthanides, actinides, and metalloids. Preferably the complex is a coordination complex bonding with at least near oxygen atoms of hydroxyl groups.

The compound may further include a ligand (or donor atom) bound to the metal.

The ligand may be a monodentate, bidentate, tridentate, tetradentate and pentadentate ligand. The donor atom can include oxygen, nitrogen, sulphur, phosphorus, and carbon.

### BRIEF DESCRIPTION OF THE DRAWINGS

Several embodiments of the invention will be described with reference to the drawings, in which:

FIG. 1 illustrates the synthesis of the multidentate ligands made conveniently stereospecific through acid-catalyzed depolymerization of oligomeric proanthocyanidins (OPCs) where Ar′=3,4-dihydroxyphenyl. R=any alkyl, alkoxy, aryl, or phenoxyl group, R₁ and R₂=any alkyl group;

FIG. 2 illustrates metal complexes formed from the chiral ligands of the embodiments, where M=any metal, particularly transition metal, lanthanides or actinides, L, represents any ligands on the metal; n=0 to 6; R₁, R₂, R₃, and R₄=any alkyl or alkoxyl group or hydrogen atom;

FIG. 3 is the IR spectra of extracted OPCs with protected groups following modification with terminal alkynes;

FIG. 4 illustrates various reaction schemes according to the embodiments for conversion of proanthocyanidins from mangsteen peels to multidentate ligands;

FIG. 5 illustrates a scheme for synthesis of a multidentate EC₂S₂ ligand derived from depolymerization of oligomeric proanthocyanidins, where Ar′=3,4-dihydroxyphenyl; and

FIG. 6 illustrates a scheme for synthesis of metal complexes of the chiral ligand shown in FIG. 5.

### DETAILED DESCRIPTION THE INVENTION

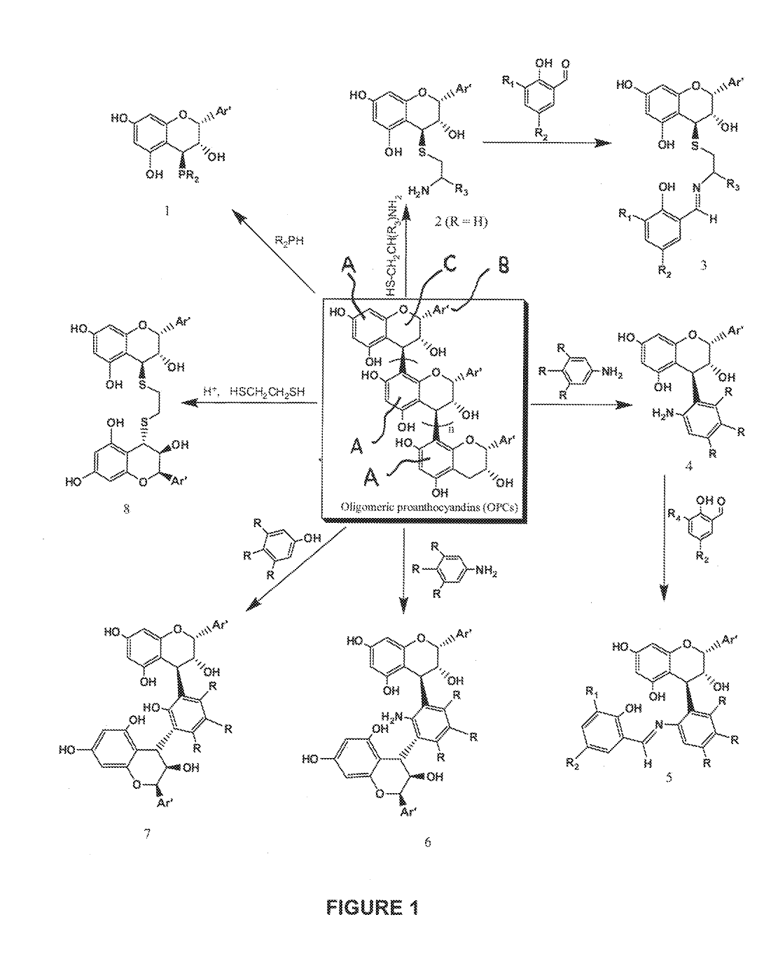

Oligomeric proanthocyanidins (OPCs) are major secondary metabolites found abundantly in plant kingdom, including those of agricultural byproducts like mangosteen pericarps, peanut skins, and grape seeds etc. OPC illustrated in FIG. 1, is typically composed of repeating units of epicatechin or catechin. It has been found that the hydroxyl groups shown on the A ring of the monomer units in the OPCs molecular structure illustrated in FIG. 1, are positioned ideally for chelating transition metals to form chiral complexes.

OPCs can be transformed either to multi-dentate chiral ligands, or immobilized in inorganic or organic polymer matrixes for ease of recycling and re-use. The potential application of OPCs as chiral auxiliary in the pharmaceutical industry and other applications have potential to impact the environment in a positive manner.

The OPC molecular structure illustrated in FIG. 1 has epicatechin as the monomeric unit, with type 4-8 linkage, including the A ring, a C ring, and a B ring in the monomeric unit. In the structure of FIG. 1, the B ring is Ar′=(HO)₂C₆H₃, and n=2 to 50. In one embodiment, with simple protection of the ortho-dihydroxyl groups on the B ring from competitive binding of metals, a new chiral ligand is obtained which can be used with effect in catalyzing organic reactions.

Mangosteen pericarps have been found to contain a large amount of OPCs that are uniformly B type linkage polymers of epicatechin with stereo regularity (Fu, C.; Loo, A. E. K.; Chia, F. P. P.; Huang, D. Oligomeric Proanthocyanidins from Mangosteen Pericarps. Journal of Agricultural and Food Chemistry (2007), 55(19), 7689-7694).

The epicatechin ligands and their metal complexes can be used in any catalytic reactions including but not limited to asymmetric hydrogenation, epoxidation, oxidation, reduction, substitution, addition, coupling, carbon carbon bond forming, carbon oxygen bond forming, carbon nitrogen bond forming reactions, kinetic resolution, carbon carbon double bond metathesis, and carbon carbon triple bond metathesis.

### EXAMPLES

### Instruments

¹H and ¹³C{¹H} NMR spectra were recorded in deuterated methanol with a Bruker AC300 spectrometer (Karlsruhe, Germany) at 300 and 75 MHz, respectively. The electrospray ionization mass spectra were obtained from a Finnigan/MAT LCQ ion trap mass spectrometer (San Jose, Calif., USA) equipped with an electrospray ionization (ESI) source. The heated capillary and voltage were maintained at 250° C. and 4.5 kV, respectively. The full-scan mass spectra from m/z 50 to 2000 were recorded.

The mangosteen pericarps proanthocyanidins were dissolved in methanol and the solution was introduced into the ion spray source with a syringe (100 μL). LC/MS spectra were acquired using Finnigan/MAT LCQ ion trap mass spectrometer (San Jose, Calif., USA) equipped with TSP 4000 HPLC system, which includes UV6000LP PDA detector, P4000 quaternary pump and AS3000 autosampler. The heated capillary and spray voltage were maintained at 250° C. and 4.5 kV, respectively. Nitrogen is operated at 80 psi for sheath gas flow rate and 20 psi for auxiliary gas flow rate. The full scan mass spectra from m/z 50-2000 were acquired both in positive and negative ion mode with a scan speed of one scan per second. MALDI-TOF mass spectra were collected on a Voyager-DE STR mass spectrometer equipped with delayed extraction and a N₂ laser set at 337 nm. The length of one laser pulse was 3 ns. The measurements were carried out using the following conditions: positive polarity, linear flight path 21 kV acceleration voltage, 100 pulses per spectrum. The samples were dissolved in methanol (4 mg/mL). Sodium chloride and 2,5-dihydroxybenzoic acid as matrix were used to enhance ion formation. Aqueous solution of sodium chloride (1.0 μL, 0.1M) was added to sample solution (1.0 mL) followed by addition of equal volume of methanol solution of 2,5-dihydroxybenzoic acid (10 mg/mL). The resulting solution (1.0 μL) was evaporated and introduced into the spectrophotometer. UV-Vis spectra were recorded using a Shimadzu UK1601 spectrophotometer fitted with a quartz cell. High resolution MS spectrum was obtained from Finnigan (MAT 95XL-T) high resolution (60,000), 5 KV Double Focusing Reversed Nier-Johnson Geometry Mass Spectrometer.

### Mean Degree of Polymerization Analysis

In a small glass vial, proanthocyanidins solution (50 μL, 2.0 mg/mL in methanol) was mixed together with methanol acidified with concentrated HCl (50 μL, 3.3%, v/v) and 100 μL of benzyl mercaptan (5% v/v in methanol). The vial was sealed with an inert Teflon cap. The reaction was carried out at 40° C. for 30 min and then kept at room temperature for 10 h; then, the reaction mixtures were kept in the freezer (−20° C.) until 10 μL was injected directly for reverse-phase HPLC analysis. The thiolysis media were further analyzed using LC/MS with a Shimadzu 250 mm×4.6 mm i.d., 5 μm C18 column (Kyoto, Japan). The binary mobile phases consisted of A (2% acetic acid in water, v/v) and B (methanol), which were delivered in a linear gradient of B from 15 to 80% (v/v) in 45 min. The flow rate was set at 1.0 mL/min.

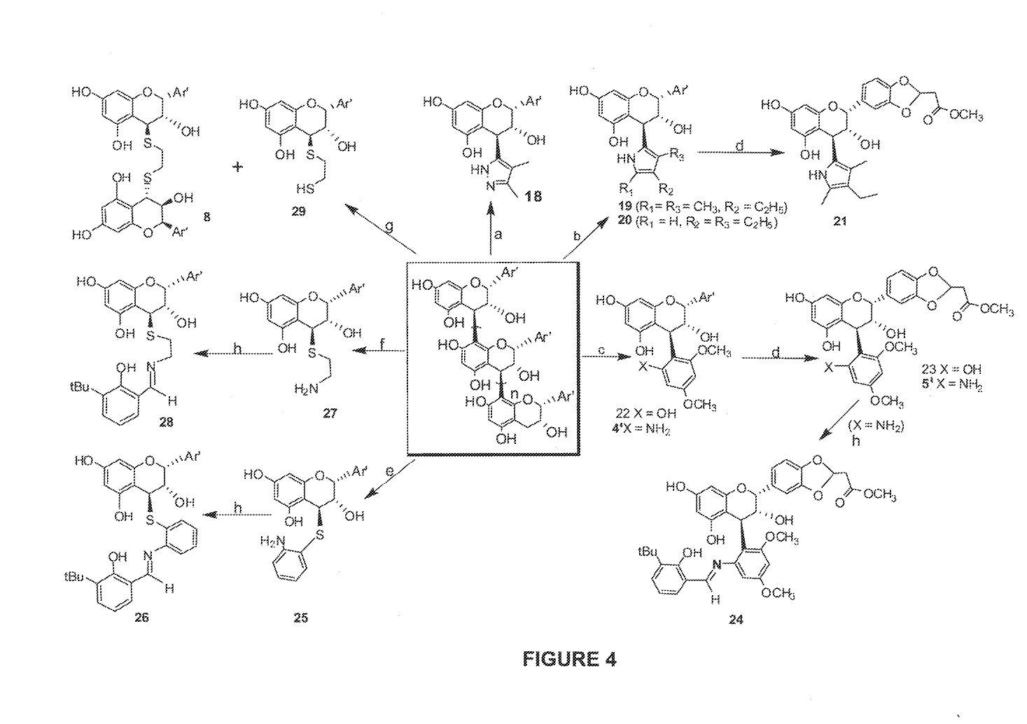

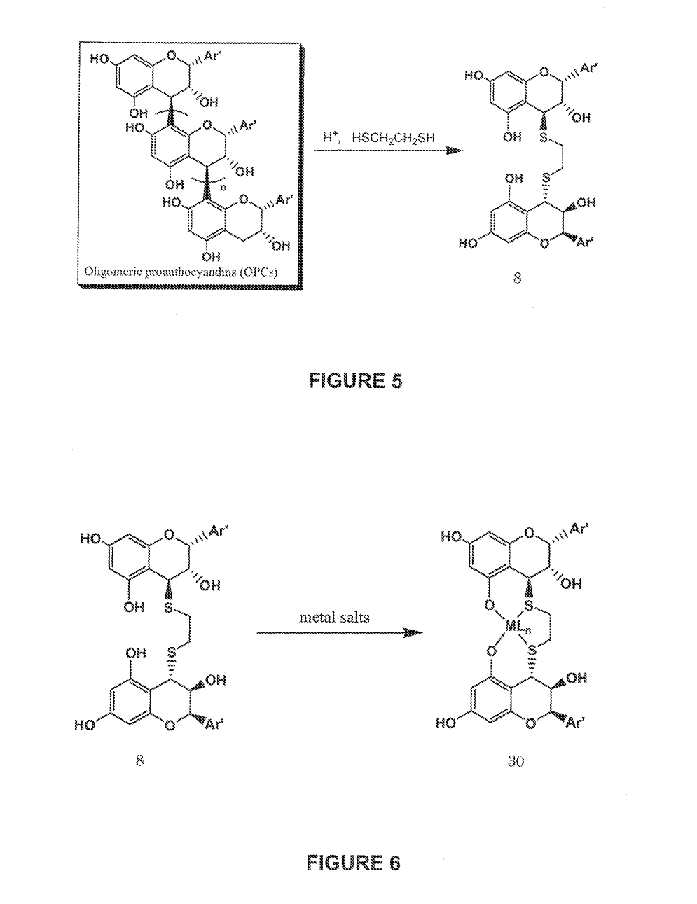

The following examples are based on the schematic reactions illustrated in FIG. 4.

### Extraction and Purification of Mangosteen Pericarp Proanthocvanidins.

OPCs isolated from mangsteen peels are an ideal source because it contains dominantly B type interflavone linkage and epicatechin as the monomeric unit with relatively high degree of polymerization.

The mangosteen pericarps (2.0 kg, fresh) were ground and Soxhlet defatted with hexane (3×1500 mL). The remaining solids were subsequently extracted by a mixture of acetone/water (7:3, 3×4000 mL) for 4 h. The mixture was filtered, and the filtrate was pooled. The acetone in the filtrate was evaporated to yield slurry, which was centrifuged at 3000 g for 15 min. The supernatant was collected and liquid-liquid extracted with dichloromethane (3×500 mL) to further remove xanthones and other lipophilic compounds. The water phase was collected and concentrated to 60 mL. The crude proanthocyanidin fraction (20 mL) was filtered through a Sartorius Minisart 45 μm porosity filter (Epsom, United Kingdom) and then loaded on a Sephadex LH-20 column containing 50 g of LH-20 equilibrated with MeOH/water (1:1) for 4 h. The column was washed with MeOH/water (1:1) until the eluent turned colorless. The adsorbed proanthocyanidins were then eluted with aqueous acetone (70%, 500 mL). The acetone was removed on a rotary evaporator at 40° C., and the resulting residue was freeze-dried to give a light brown powder (4.2 g overall yield). The moisture content in mangosteen was determined to be 68.3%, and thus, the yield of the oligomeric proanthocyanidins (Proanthocyanidins) was 0.66% of dry matter. The purity measured by UV/vis colorimetric methods analysis showed that the extract contains over 99% (wt) epicatechin (standard) equivalents.

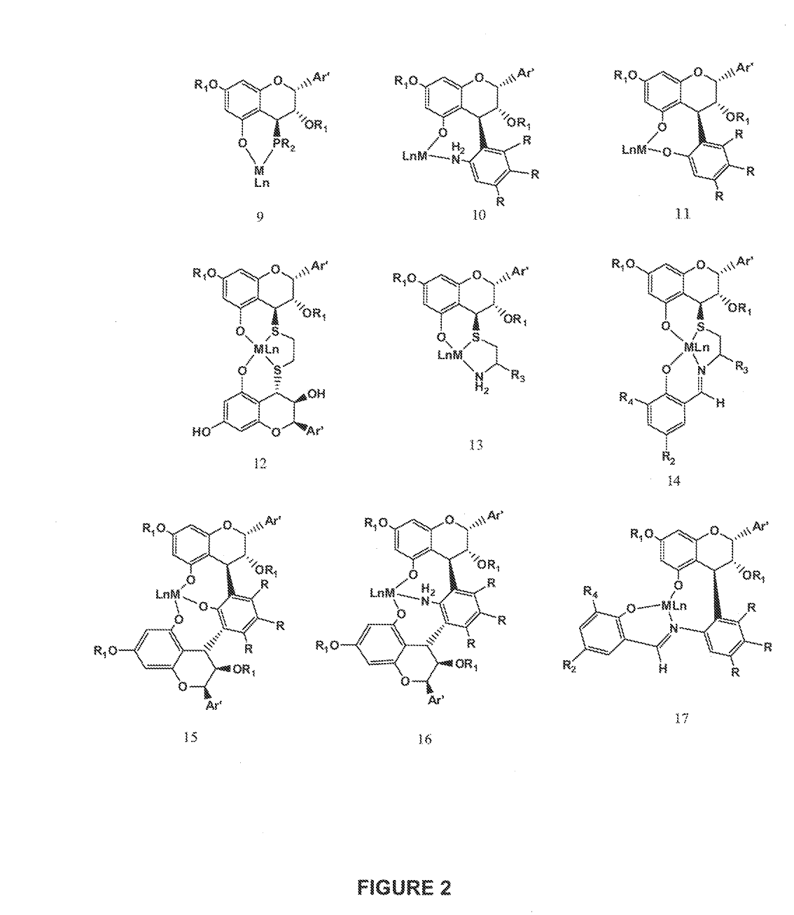

Following the extraction and purification, the OPCs were treated to produce derivatives thereon. The OPCs were generally depolymerized with a nucleophile in the presence of an acid. The following examples delineate the approach of:—
1. Protecting the hydroxyl groups in the repeat units of the OPCs; and
2. Depolymerization using nucleophiles
   to form intermediary products (e.g., multi-dentate ligands/other derivatives) from which, for example, metal complexes such as those illustrated in FIG. 2 can be synthesized. Further from these intermediary products additional compounds can be derived.
   I. Protection of the HO Groups in Proanthocyanidins with Terminal Alkynes.

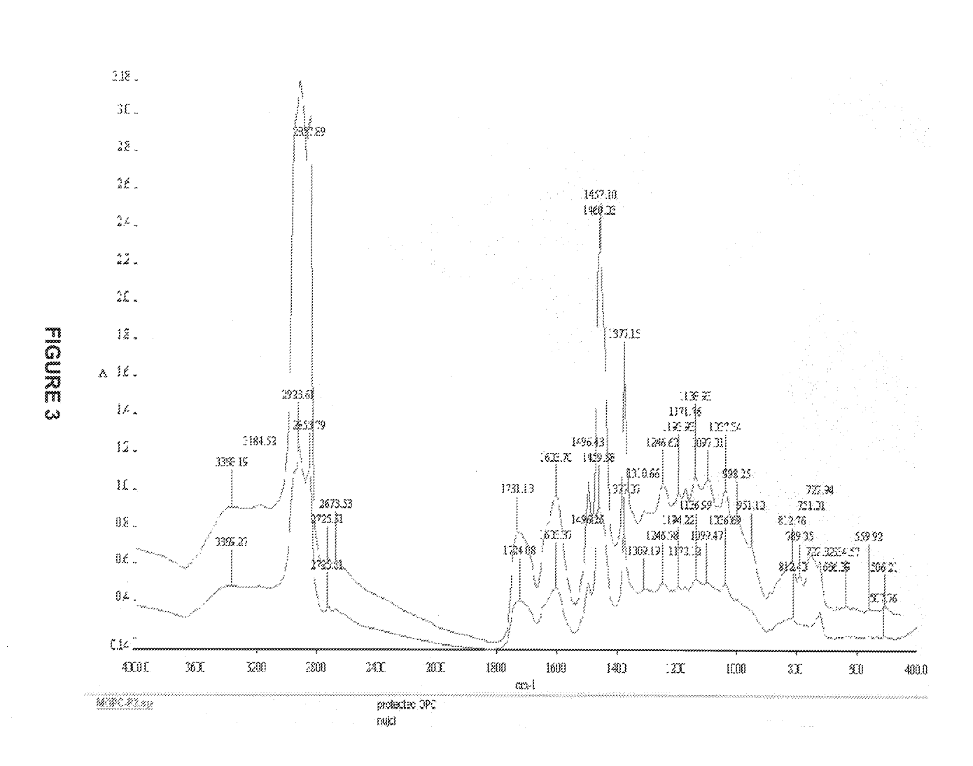

Oligomeric proanthocyandin (2.0 g, isolated from mangosteen pericarp) was dissolved in DMSO (25.0 mL-250 mL). To the solution, propynoate methyl ester (0.64 g) and N,N-dimethylpyridine (DMAP, 0.843 g) were added. The mixture was stirred at room temperature for one week and extracted with diethyl ether after 1.0 mL acetic acid was added to quench the reaction. The diethyl ether extract was dried over sodium sulphate and the volatiles were removed to yield small amount of residue and was discarded. The DMSO solution was precipitated into water to give dark brown solid. The solid and the solution were extracted with ethyl acetate three times (overall 100 mL), the ethyl acetate layer was washed with water three times and dried over sodium sulphate. The volatiles were evaporated to give brown solid which was washed with diethyl ether three times and dried under vacuum. The product is coded as MOPC-P. The residue that is not soluble in water and ethyl acetate was washed with water multiple times and dried in vacuum overnight to give 1.0 gram of powder labelled MOPC-P2. The IR and NMR spectra show desired product were obtained and are illustrated in FIG. 3, with the lower line being the IR spectrum for MOPC-P, and the upper line being the IR spectrum of MOPC-P2.

The following diagram shows the representative structure of MOPC-P.

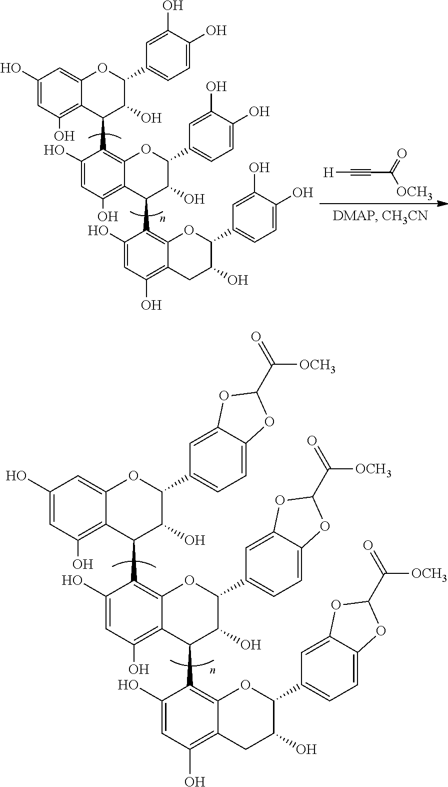

### II. Depolymerization of OPCs

By selecting proper carbon and thiol nucleophiles, the inventors were able to obtain a number of novel epicatechin derivatives by acid depolymerization of mangosteen OPCs. FIG. 4 illustrates the various reaction schemes of acid depolymerization of OPCs.

FIG. 4 illustrates several reaction schemes of the conversion of oligomeric proanthocyanidins (OPCs) from mangosteen peel to multidentate ligands. Ar′=3,4-dihydroxylphenyl. Acid depolymerization conditions are 0.22% HCl in methanol 45° C., 2 to 8 hours. Reaction scheme a, in the presence of 2,3-dimethylpyrazole produces compound 18. Reaction scheme b in the presence of 3-ethyl-2,4-dimethylpyrrole produces compound 19; or in the presence of 3,4-diethylpryrrole produces compound 20. Reaction scheme c, in the presence of 3,5-dimethoxyphenol produces compound 22; or in the presence of 3,5-dimethoxyaniline produces compound 4′. Thereafter with reaction scheme d in the presence of 4-(N,N-dimethylamino)pyridine, HC≡CCOOCH₃, CH₃CN, rt. 12 hrs, produces compound 21, 23 or 5′, respectively. Reaction scheme e in the presence of o-thioaniline produces compound 25. Reaction scheme f in the presence of cysteamine produces compound 27. Reaction scheme g in the presence of 1,2-dithioethane produces compound 29 and compound 8. Following from reaction scheme d, reaction scheme e, and reaction scheme f, with reaction scheme h in the presence of 3-tert-butyl-salicylaldehyde, CH₃OH, AcOH, 1 drop, rt, produces compound 24, 26, and compound 28, respectively. Referring to FIG. 4, compounds 18, 19 and 20 were isolated by normal phase silica column chromatography with satisfactory yield. In contrast, when pyrrole was used as nucleophile black mixtures were obtained possibly due to polymerization reaction. Compounds 18, 19, and 20 are rare flavonoidal alkaloids. Naturally, there are three reported case of such compounds, lotthanongine (flavonoidal indole derivative) (Kanchanapoom, T.; Kasai, R.; Chumsri, P.; Kraisintu, K.; Yamasaki, K. Lotthanongine, an unprecedented flavonoidal alkaloid from the roots of That medicinal plant, Trigonostemon reidioides. Tetrahedron Lett. 2002, 43, 2941-2943.), ficine/isoficine (Johns, S. R.; Russel, J. H.; Hefferman, M. L. Ficine, A novel flavonoidal alkaloid from Tetrahedron Lett. 1965, 24, 1987-1991.), and phyllospadine (Takagi, M.; Funahashi, S.; Ohta, K.; Nakabayashi, T. Phyllospadine, a New Flavonoidal Alkaloid from the Sea-Grass Phyllosphadix iwatensis. Agric. Biol. Chem. 1980, 44, 3019-3020.). The bioactivity of these compounds remains largely unexplored. Compounds 18, 19 and 20 have two metal chelating sites and are potential as ligands to prepare bimetallic catalysts. The HPLC chromatograms of the compounds all gave rise to one sharp peak indicating a single enantiomer for Compounds 18, 19 and 20.

The o-dihydroxyl group on B ring (FIG. 2) can be blocked selectively with methyl propiolate in the presence of 4-N,N-dimethylaminopyridine (DMAP) under mild conditions. For example, compounds 21 and 23 were made this way. They exist as two diastereomers due to two equally populated chiral configuration at O—C*—O indicated by two equal intensity ¹H NMR resonance signals around 6.52-6.48 ppm. Compound 23 can be further converted to compound 24 in high yield through reaction with 3-tert-butyl-2-hydroxybenzaldehyde which has a chiral tridentate pocket for metal ions.

Using thiol as nucleophiles, compounds 25, 27, 29 and 8 were also prepared. Conversion of compound 25 to compound 26, and conversion of compound 27 to compound 28 were readily accomplished.

General Procedure for the Acid Depolymerization of OPCs in the Presence of Carbon and Sulfur Nucleophiles. Synthesis of the chiral Ligands 4, 18, 19, 20, 22, 25, 27, 29, and 30.

Under nitrogen atmosphere, the mangosteen OPCs (9.0 g) was mixed with MeOH (2 00 mL), hydrochloric acid (36%, 2 mL), and nuclephiles. The mixture was heated at 50° C. for 8 hrs with stirring. The filtrate was neutralized with 0.1M NaHCO₃ to pH 7.0 before it was extracted with ethyl acetate. The combined organic fraction was dried over anhydrous sodium sulphate. Evaporation of the ethyl acetate gave dark brown residue, which was purified with column chromatography (detailed conditions were described under individual compounds) to afford the chiral ligands

### General Procedure for Selective Protection of the Ortho-Dihydroxyl Groups in Chiral Ligands 4, 19, and 22:

Under nitrogen atmosphere, a 50 mL acetonitrile solution of chiral ligand 4 (1 mmol) and methyl propiolate (1.1 mmol) was added 4-N-dimethylaminopyridine (DMAP) (1.5 mmol). The mixture was stirred at room temperature for 8 h. The volatiles were removed under reduced pressure and the residue was purified by column chromatography on silica gel to afford the chiral ligand 5. Similarly, chiral ligand 19 is converted to chiral ligand 21, and chiral ligand 22 is converted to chiral ligand 23.

### General Procedure the Preparation of Schiff Bases 24, 26, 28.

To a solution of chiral ligand 5 (1 mmol) in MeOH (5 mL) was successively added 3-tert-butyl-2-hydroxybenzaldehyde (1.1 mmol) and one drop of acetic acid. The reaction mixture was refluxed for 8 h and the solvent was then removed under reduced pressure. The residue was purified by further to afford chiral ligand 24. In similar fashion, chiral ligand 26 was prepared from chiral ligand 25, and chiral ligand 28 from chiral ligand 27.

### III. Spectral Data of the Compounds:

### Compound 18

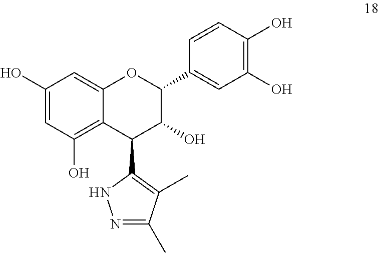

(2R,3R,4R)-2-(3′,4′-dihydroxyphenyl)-4-(3″,4″-dimethyl-1H-pyrazol-5-yl)chroman-3,5,7-triol was purified with column chromatography (silica gel, EtOAc-hexanes 2:1 and then dichloromethane-methanol 8:1) as a yellow solid. MS (ESI, 383 [M−H]⁻. ¹H-NMR (300 MHz, acetone-d₆): δ=6.86 (d, 1H, C(2′)-H, J=1.8), 6.73 (d, 1H, C(5′)-H, J=8.6), 6.65 (d, 1H, C(6′)-H, J=8.6), 6.04 (d, 1H, C(6)-H, J=2.3), 6.03 (d, 1H, C(8)-H, J=2.3), 5.34 (d, 1H, C(2)-H, J=2.3), 4.59 (s, 1H, C(4)-H), 4.22 (brs, 1H, C(3)-H), 2.18 (s, 3H, —CH₃), 1.94 (s, 3H, —CH₃). ¹³C{¹H}NMR (75 MHz, acetone-d6): δ 159.2, 158.4, 157.0, 147.4, 144.5, 129.6, 128.1, 117.8, 114.5, 113.8, 112.8, 95.7, 95.2, 94.3, 74.1, 69.5, 56.8, 10.0, 6.9. IR (KBr): 3368, 2969, 1619, 1519, 1448, 1283, 1154, 1109, 1075, 842, 795, 764, 668, 630, 535 cm⁻¹.

### Compound 19

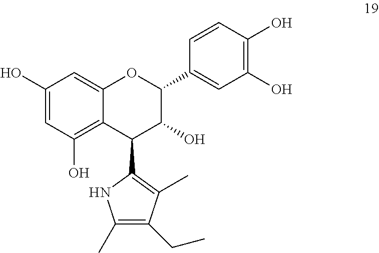

(2R,3R,4R)-2-(3,4-dihydroxyphenyl)-4-(4-ethyl-3,5-dimethyl-1H-pyrrol-2-yl)chroman-3,5,7-triol was purified with column chromatography (silica gel, EtOAc-hexanes 2:1 and then dichloromethane-methanol 9:1) as a red solid. MS (ESI, −c): 410 [M−H]⁻. ¹H-NMR (300 MHz, acetone-d₆): δ=6.97 (s, 1H, C(2′)-H), 6.76 (d, J=8.2, 1H, C(6′)-H), 6.71 (d, J=8.2 Hz, 1H, C(5′)-H), 6.00 (s, 1H, C(6)-H), 5.98 (s, 1H, C(8)-H), 4.81 (s, 1H, C(2)-H), 4.29 (s, 1H, C(4)-H), 3.98 (s, 1H, C(3)-H), 2.36 (dd, J₁=7.3, J₂=7.5, 2H, C(10)-H), 2.06 (s, 3H, C(9)-H), 1.99 (s, 3H, C(12)-H), 1.03 (t, J=7.5, 3H, C(11)-H). ¹³C{¹H}NMR (75 MHz, acetone-d₆): δ 157.41, 157.14, 156.64, 144.44, 144.25, 131.26, 125.45, 120.63, 120.03, 114.61, 111.77, 99.31, 95.67, 94.85, 74.91, 71.4, 37.15, 17.39, 15.30, 9.98, 8.55. IR (KBr): 3367, 2968, 1619, 1519, 1497, 1446, 1374, 1284, 1153, 1108, 1062, 1021, 822, 767, 672, 544 cm⁻¹.

### Compound 20

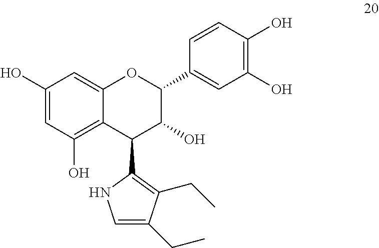

(2R,3R,4R)-4-(3,4-diethyl-1H-pyrrol-2-yl)-2-(3,4-dihydroxyphenyl)chroman-3,5,7-triol was purified with column chromatography (silica gel, EtOAc-hexanes 2:1 and then dichloromethane-methanol 9:1) as a red solid. MS (ESI, −c): 410 [M−H]⁻. ¹H-NMR (300 MHz, CD₃CN): δ=6.90 (s, 1H, C(2′)-H), 6.76 (d, J=8.2, 1H, C(6′)-H), 6.72 (d, J=8.2 Hz, 1H, C(5′)-H), 5.98 (s, 1H, C(6)-H), 5.92 (s, 1H, C(8)-H), 5.30 (s, 1H, C(2)-H), 4.18 (s, 1H, C(4)-H), 4.16 (s, 1H, C(3)-H), 2.39 (m, 4H, —CH2), 1.08 (t, J=7.5, 3H, —CH₃), 1.00 (t, 3H J=7.5, 3H, —CH₃).

### Compound 21

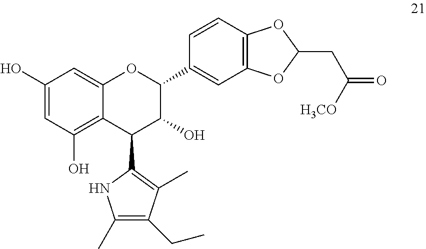

Methyl 2-(5-((2R,3R,4R)-4-(4-ethyl-3,5-dimethyl-1H-pyrrol-2-yl)-3,5,7-tri hydroxychroman-2-yl)benzo[d][1,3]dioxol-2-yl)acetate was purified with column chromatography (silica gel, dichloromethane-methanol 13:1) as a red solid. MS (ESI, −c): 494 [M−H]⁻. ¹H-NMR (300 MHz, acetone-d₆): δ 7.02 (s, 1H, C(2′)-H), 6.77 (s, 2H, C(5′)-H, C(6′)-H), 6.50 (t, J=5.3, 1H, C(13)-H), 6.01 (d, J=2.4, 1H, C(6)-H), 5.97 (d, J=2.4, 1H, C(8)-H), 4.85 (s, 1H, C(2)-H), 4.29 (s, 1H, C(4)-H), 3.97 (s, 1H, C(3)-H), 3.65 (s, 3H, C(16)-H), 3.04 (dd, J₁=1.5, J₂⁼3.8, 2H, C(14)-H), 2.35 (dd, J=7.5, J₂=7.5, 2H, C(10)-H), 2.06 (s, 3H, C(9)-H), 1.98 (s, 3H, C(12)-H), 1.29 (s, 3H, C(11)-H). ¹³C{¹H}NMR (75 MHz, acetone-d₆): δ 164.5, 151.4, 152.6, 148.7, 131.8, 129.9, 122.7, 121.8, 110.1, 109.3, 109.2, 97.7, 96.6, 92.6, 83.0, 75.5, 52.8, 41.1, 25.6, 20.8, 13.8, 8.8, 5.9. IR (KBr): 3332, 2973, 2934, 1694, 1497, 1440, 1376, 1314, 1252, 1153, 1106, 1048, 991, 840, 765, 698, 633, 546 cm⁻¹.

### Compound 22

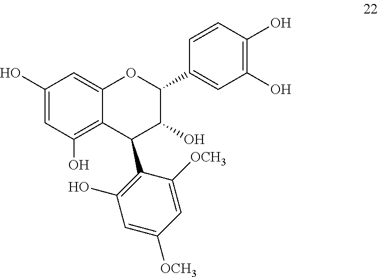

(2R,3R,4S)-2-(3,4-dihydroxyphenyl)-4-(2-hydroxy-4,6-dimethoxyphenyl)-3,4-dihydro-2H-chromene-3,5,7-triol was purified with column chromatography (silica gel, EtOAc-hexanes 2:1 and then dichloromethane-methanol 8:1) as a light yellow solid. MS (ESI, −c): 441 [M−H]⁻. ¹H-NMR (300 MHz, acetone-d₆): δ=6.98 (d, J=1.8 Hz, 1H, C(2′)-H), 6.75 (d, J=8.0, 1H, C(6′)-H), 6.72 (d, J=1.8 Hz, 1H, C(5′)-H), 5.97 (s, 1H, C(6)-H), 5.91 (m, 3H, C(8)-H, C(3″)-H, C(5″)-H), 5.04 (s, 1H, C(2)-H), 4.61 (s, 1H, C(4)-H), 3.91 (s, 1H, C(3)-H), 3.74 (s, 6H, OCH₃). ¹³C{¹H}NMR (75 MHz, acetone-d₆): δ 161.18, 158.33-158.81, 146.06, 145.84, 133.10, 119.81, 116.15, 115.84, 96.79, 96.05, 92.85, 77.51, 73.35, 56.91, 55.95, 37.38. IR: 3391, 2938, 1615, 1516, 1466, 1361, 1282, 1202, 1146, 1092, 1056, 1018, 818, 632, 540 cm⁻¹. HRMS: calcd. for C₂₃H₂₁O₉ 441.1180; found 441.1190.

### Compound 4

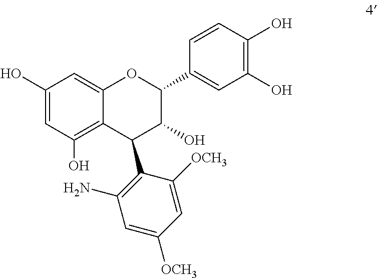

(2R,3R,4S)-4-(2-amino-4,6-dimethoxyphenyl)-2-(3,4-dihydroxyphenyl)chroman-3,5,7-triol (4) was purified with column chromatography (silica gel, EtOAc-hexanes 2:1 and then dichloromethane-methanol 9:1) as a light brown solid. MS (ESI, −c): 440 [M−H]⁻. ¹H-NMR (300 MHz, acetone-d₆): δ=6.98 (d, J=1.8 Hz, 1H, C(2′)-H), 6.75 (d, J=8.0, 1H, C(6′)-H), 6.72 (d, J=1.8 Hz, 1H, C(5′)-H), 5.97 (s, 1H, C(6)-H), 5.91 (m, 3H, C(8)-H, C(3″)-H, C(5″)-H), 5.04 (s, 1H, C(2)-H), 4.61 (s, 1H, C(4)-H), 3.91 (s, 1H, C(3)-H), 3.74 (s, 6H, OCH₃). ¹³C{¹H}NMR (75 MHz, acetone-d₆): δ=159.6 (2C, C-2″, C-6″), 155.6-156.1 (4C, C-5, C-7, C-8a, C-4″), 144.1, 143.8 (C-3′, C-4′), 131.0 (C-1′), 127.9 (C-6′), 118.4 (C-5′), 114.7 (C-2′), 113.9 (C-4-a), 95.2-94.2 (4C, C1″, C5″, C6, C8), 89.1 (C-3″), 76.4 (C-2), 71.9 (C-3), 55.4 (C-10), 54.5 (C-9), 36.1 (C-4). IR (KBr): 3368, 2938, 2841, 1607, 1516, 1465, 1341, 1283, 1244, 1204, 1150, 1116, 1091, 1061, 1018, 933, 821, 792, 667, 632, 542, 494 cm⁻¹

### Compound 23

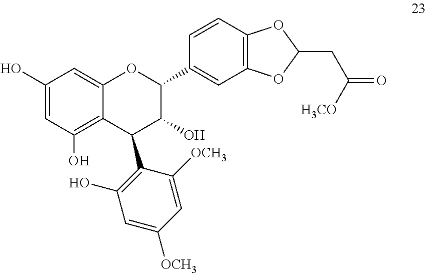

Methyl 2-(5-((2R,3R,4S)-3,5,7-trihydroxy-4-(2-hydroxy-4,6-dimethoxyphenyl)chroman-2-yl)benzo[d][1,3]dioxol-2-yl)acetate was purified with column chromatography (silica gel, EtOAc-hexanes 3:2) as a white solid. MS (ESI, −c): 525 [M−H]⁻. ¹H-NMR (300 MHz, acetone-d₆): δ=7.06 (s, 1H, C(2′)-H), 6.79 (d, J=8.0, 1H, C(6′)-H), 6.79 (d, J=8.0 Hz, 1H, C(5′)-H), 6.51 (t, J=5.3 Hz, 1H, C(11)-H), 6.12 (s, 1H, C(6)-H), 6.01 (s, 3H, C(6)-H, C(3″)-H, C(5″)-H), 5.10 (s, 1H, C(2)-H), 4.62 (s, 1H, C(4)-H), 3.90 (s, 1H, C(3)-H), 3.72 (m, 9H, OCH3), 3.01 (dd, J=0.8, J₂=5.3, 2H, C(12)-H). ¹³C{¹H}NMR (75 MHz, acetone-d₆): δ 169.83, 162.83, 161.25, 158.21-159.42, 148.45, 147.88, 135.89, 121.74, 109.00-109.71, 96.82, 96.11, 92.81, 77.64, 73.34, 56.94, 55.96, 52.78, 41.09, 37.47. IR (KBr): 3415, 3001, 2953, 2843, 1736, 1619, 1498, 1465, 1442, 1363, 1316, 1251, 1203, 1174, 1148, 1094, 1049, 1038, 949, 857, 816, 789, 754, 535, 537, 497 cm⁻¹.

### Compound 5

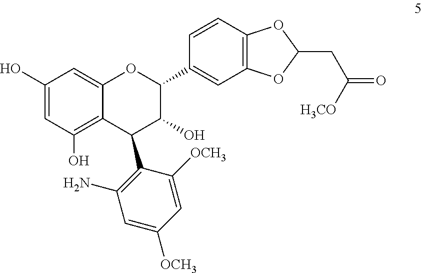

Methyl 2-(5-((2R,3R,4S)-4-(2-amino-4,6-dimethoxyphenyl)-3,5,7-trihydroxy chroman-2-yl)benzo[d][1,3]dioxol-2-yl)acetate was purified with column chromatography (silica gel, dichloromethane-methanol 11:1) as a yellow solid. MS (ESI, −c): 524 [M−H]⁻. ¹H-NMR (300 MHz, acetone-d₆): δ=6.95 (s, 1H, C(2′)-H), 6.76 (d, J=8.1, 1H, C(6′)-H), 6.74 (d, J=8.1 Hz, 1H, C(5′)-H), 6.47 (m, 1H, C(11)-H), 6.02 (s, 1H, C(6)-H), 6.01 (s, 1H, C(6)-H), 5.96 (C(3″)-H), 5.82 (C(5″)-H), 5.07 (s, 1H, C(2)-H), 4.59 (s, 1H, C(4)-H), 3.81 (s, 4H, C(3)-H, C(13)-H), 3.72 (s, 6H, C(14)-H, C(15)-H), 2.98 (dd, J₁=2.1, J₂=3.0, 2H, C(12)-H). ¹³C{¹H}NMR (75 MHz, acetone-d₆): δ 169.81, 161.06, 158.99, 158.03, 157.75, 157.60, 148.40, 147.83, 135.75, 121.63, 109.67, 109.61, 109.46, 108.98, 96.96, 96.45, 96.21, 95.85, 77.28, 73.82, 55.72, 52.78, 41.04, 36.84. IR (KBr): 3368, 2936, 2841, 1731, 1607, 1497, 1440, 1236, 1202, 1147, 1036, 812, 788, 753, 631, 537 cm⁻¹.

### Compound 24

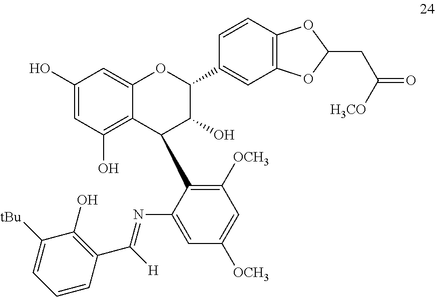

Methyl 2-(5-((2R,3R,4S)-4-(2-((E)-3-tert-butyl-2-hydroxybenzylideneamino)-4,6-dimethoxyphenyl)-3,5,7-trihydroxychroman-2-yl)benzo[d][1,3]dioxol-2-yl)acetate was purified via washing with hexane as a yellow solid. MS (ESI, −c): 684 [M−H]⁻. ¹H-NMR (300 MHz, acetone-d6): δ=8.96 (s, 1H, C(16)-H), 7.43 (s, 1H, C(18)-H), 7.40 (s, 1H, C(20)-H), 7.08 (s, 1H, C(2′)-H), 6.90 (t, J=7.7, C(19)-H), 6.76 (m, 3H, C(5′), C(6′), C(13)-H), 6.51 (t, J=2.3, 2H, 2H, C(11), C(23)-H), 6.00 (d, J=2.3, 1H, C(6)-H), 5.96 (d, J=2.3, 1H, C(8)-H), 5.18 (C(2)-H), 4.75 (C(4)-H), 3.89 (s, 1H, C(3)-H), 3.84 (s, 3H, —OCH₃), 3.70 (s, 6H, —OCH₃), 3.01 (d, J=5.3, C(24)-H), 1.46 (s, 9H, -tBu). ¹³C{1H}NMR (75 MHz, acetone-d6): δ 168.13, 163.74, 160.30, 156.44, 156.17, 147.82, 146.92, 145.03, 134.48, 131.02, 130.00, 120.03, 118.30, 108.3, 107.87, 107.32, 94.74, 94.27, 75.96, 71.65, 55.78, 51.10, 39.43, 36.01, 34.42, 31.16. IR (KBr): 3368, 2936, 2841, 1731, 1607, 1497, 1440, 1236, 1202, 1147, 1036, 812, 788, 753, 631, 537 cm⁻¹.

### Compound 25

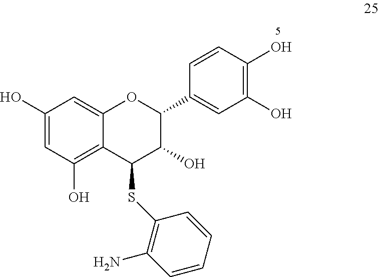

(2R,3S,4S)-4-(2-aminophenylthio)-2-(3,4-dihydroxyphenyl)chroman-3,5,7-triol was purified with column chromatography (silica gel, EtOAc-hexanes 2:1 and then dichloromethane-methanol 8:1) as a yellow solid. MS (ESI, −c): 412 [M−H]⁻. ¹H-NMR (300 MHz, acetone-d₆): δ=7.41 (dd, J=1.7, 1.7, 1H, C(13)-H), 6.77 (d, J=1.7, 1H, C(2′)-H), 7.12 (dd, J=0.7, 1.7, 1H, C(11)-H), 6.94 (dd, J=1.5, 1.7, 1H, C(12)-H), 6.86 (m, 2H, C(5′)-H, C(6′)-H), 6.57 (m, C(10)-H), 6.60 (d, J=2.3, 1H, C(6)-H), 6.00 (d, J=2.3, 1H, C(8)-H), 5.66 (s, 1H, C(2)-H), 4.34 (s, 1H, C(4)-H), 3.94 (s, 1H, C(3)-H). ¹³C{¹H}NMR (75 MHz, acetone-d₆): δ 159.80, 159.32, 158.38, 152.29, 146.20, 146.04, 138.74, 132.78, 132.01, 119.98, 118.06, 116.68, 116.33, 116.13, 115.98, 99.91, 97.36, 996.65, 75.79, 71.12, 45.25. IR (KBr): 3360, 1692, 1607, 1517, 1476, 1447, 1367, 1283, 1187, 1148, 1092, 1062, 1040, 1017, 973, 852, 821, 787, 754, 704, 671, 537 cm⁻¹.

### Compound 26

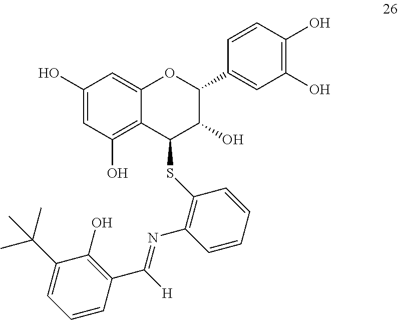

(2R,3S)-4-(2-((E)-3-tert-butyl-2-hydroxybenzylideneamino)phenylthio)-2-(3, 4-dihydroxyphenyl)chroman-3,5,7-triol was purified with column chromatography (silica gel, dichloromethane-methanol 12:1) as a yellow solid. MS (ESI, −c): 572 [M−H]⁻. ¹H-NMR (300 MHz, acetone-d₆): δ=8.85 (s, 1H, C(13)-H), 7.92 (d, J=2.6, 1H, C(14)-H), 7.43 (m, 3H, C(9, 10, 16)-H), 7.36 (m, 2H, C(11, 12)-H), 7.12 (s, 1H, C(2′)-H), 6.92 (t, 1H, C(15)-H), 6.79 (d, J=8.1, 1H, C(5′)-H), 6.71 (d, J=8.1, 1H, C(6′)-H, 6.15 (d, J=2.2, 1H, C(6)-H), 6.03 (d, J=2.2, 1H, C(8)-H), 5.60 (s, 1H, C(2)-H), 4.82 (s, 1H, C(4)-H), 4.01 (s, 1H, C(3)-H), 1.44 (s, 9H, tBu). ¹³C{¹H}NMR (75 MHz, acetone-d₆): δ 164.12, 158.43, 157.85, 156.80, 147.32, 144.48, 137.05, 130.78, 129.79, 122.31, 118.62, 114.55, 114.54, 97.31, 95.91, 94.91, 74.67, 65.97, 44.85, 34.49, 29.65. IR (KBr): 3367, 2957, 1697, 1607, 1520, 1441, 1364, 1282, 1197, 1146, 1100, 1061, 1018, 973, 855, 821, 795, 751, 670, 540 cm⁻¹.

### Compound 27

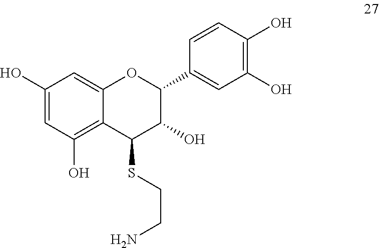

(2R,3S,4S)-4-(2-aminoethylthio)-2-(3,4-dihydroxyphenyl)chroman-3,5,7-triol was purified with reversed phased column chromatography (gradient elution of MeOH/H₂O (1:4, v/v), 0.4% aq. AcOH/MeOH (4:1, v/v), 0.4% aq. AcOH/MeOH (1:1, v/v)). The pure fractions were combined, neutralized using pH 6.5 Na₂HPO₄ buffer, and then concentrated. After extraction with acetone, the extract was dried in vacuum to furnish 15 as a red solid. MS (ESI, +c): 366 [M+H]⁺. ¹H-NMR (300 MHz, D₂O): δ=6.92 (d, 1H, C(2′)-H), 6.82-6.77 (m, 2H, C(5′)-H, and C(6′)-H), 6.01 (d, J=2.3 Hz, 1H, C(8)-H), 5.95 (d, J=2.3 Hz, 1H, C(6)-H), 5.14 (s, 1H, C(2)-H), 4.00 (d, J=2.3 Hz, 1H, C(4)-H), 3.86 (d, J=2.3 Hz, 1H, C(3)-H), 3.30-2.80 (m, 4H, S—CH₂—CH₂—N); ¹³C{¹H}-NMR (75 MHz, D₂O): δ=156.6, 156.2, 155.1, 143.7, 143.5, 130.3, 118.8, 115.9, 114.2, 98.7, 96.1, 95.2, 73.9, 70.1, 40.6, 38.4, 28.7. IR (KBr): 3352, 3189, 1620, 1519, 1468, 1384, 1284, 1192, 1150, 1094, 1063, 1017, 988, 888, 825, 783, 656, 540, 482 cm⁻¹.

### Compound 28

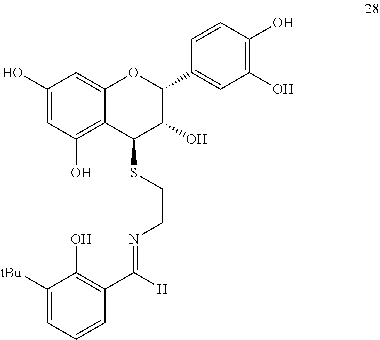

(2R,3S,4S)-4-(2-((E)-3-tert-butyl-2-hydroxybenzylideneamino)ethylthio)-2-(3, 4 dihydroxyphenyl)chroman-3,5,7-triol was purified via washing with hexane as a yellow solid. MS (ESI, +c): 526 [M−H]⁺. ¹H-NMR (300 MHz, (CD₃)₂C0) δ 8.59 (s, 1H, C(13)-H), 7.63-7.23 (m, 2H, C(6″)-H and C(4″)-H), 7.11 (s, 1H, C(5″)-H), 6.87-6.79 (m, 3H, C(6′)-H, C(5′)-H and C(2′)-H), 6.05 (s, 1H, C(8)-H), 5.92 (s, 1H, C(6)-H), 5.34 (s, 1H, C(2)-H), 4.18 (d, J=2.1 Hz, 1H, C(4)-H), 4.12 (s, 1H, C(3)-H), 3.87 (m, 2H, C(11)-H), 3.21-3.00 (m, 2H, C(10)-H), 1.42 (s, 9H, (CH₃)₃). ¹³C{¹H}-NMR (75 MHz, (CD₃)₂CO): 167.4, 157.9, 157.4, 156.2, 144.5, 144.4, 136.6, 131.0, 130.0, 118.8, 118.4, 117.8, 114.6, 114.4, 99.0, 95.6, 94.7, 74.4, 70.7, 58.5, 42.1, 34.3, 32.7. IR (KBr): 3370, 2957, 2739, 1703, 1610, 1518, 1437, 1375, 1281, 1198, 1145, 1092, 1062, 823, 753, 679, 545, 483 cm⁻¹.

### Compound 29

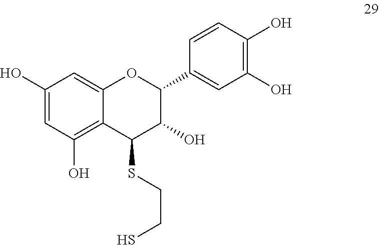

(2R,3S,4S)-2-(3,4-dihydroxyphenyl)-4-(2-mercaptoethylthio)chroman-3,5,7-triol was purified with column chromatography (silica gel, EtOAc-hexanes 2:1 and then dichloromethane-methanol 9:1) as a red solid. MS (ESI, −c): 381 [M−H]⁻. ¹H-NMR (300 MHz, CD₃OD): δ=6.99 (d, J=1.65 Hz, 1H, C(2′)-H), 6.83 (dd, J₁=1.65 Hz, J₂=8.0 Hz, 1H, C(6′)-H), 6.78 (d, J=8.0 Hz, 1H, C(5′)-H), 5.96 (d, J=2.31 Hz, 1H, C(8)-H), 5.90 (d, J=2.31 Hz, 1H, C(6)-H), 5.26 (s, 1H, C(2)-H), 4.02 (d, J=2.31 Hz, 1H, C(4)-H), 3.98 (dd, J₁=0.9 Hz, J₂=2.31 Hz, 1H, C(3)-H), 2.80-3.08 (m, 4H, SCH₂CH₂S). ¹³C{¹H}NMR (75 MHz, CD₃COCD₃): δ 159.17, 158.67, 157.34, 145.76, 145.68, 132.19, 119.62, 115.84, 115.63, 100.20, 96.90, 95.93, 75.56, 72.04, 43.32, 37.32, 25.88. IR (KBr): 3369, 1626, 1516, 1470, 1280, 1146, 1091, 1059, 1016, 852, 821, 783 cm⁻¹.

### Compound 8

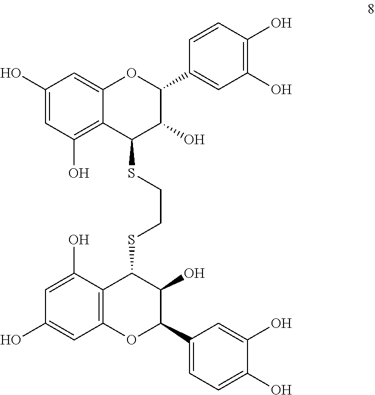

(2R,2′R,3S,3′S,4S,4′S)-4,4′-(ethane-1,2-diylbis(sulfanediyl))bis(2-(3, 4-dihydroxyphenyl)chroman-3,5,7-triol) was purified with column chromatography (silica gel, dichloromethane-methanol 5:1) as a red solid. MS (ESI, −c): 669 [M−H]⁻. ¹H-NMR (300 MHz, CD₃OD): δ=7.01 (d, J=1.8 Hz, 2H, C(2′)-H), 6.83 (dd, J₁=1.8 Hz, J₂=8.0 Hz, 2H, C(6′)-H), 6.78 (d, J=8.0 Hz, 2H, C(5′)-H), 5.96 (s, 2H, C(8)-H), 5.91 (s, 2H, C(6)-H), 5.28 (s, 2H, C(2)-H), 4.60, (s, 2H, C(4)-H), 4.05 (s, 2H, C(3)-H), 3.11 (dq, J=15.6 Hz, 4H, SCH₂CH₂S). ¹³C NMR (75 MHz, CD₃OD): δ 159.08, 158.8, 157.16, 146.00, 145.81, 132.05, 119.36, 116.05, 115.33, 100.30, 96.85, 95.75, 75.63, 72.26, 43.68, 33.94. IR (KBr): 3392, 1610, 1519, 1445, 1373, 1283, 1148, 1099, 1062, 820 cm⁻¹.

Extraction and Purification of Pine Bark Proanthocyanidins, Production of EC₂S₂ Ligands and Preparation and Analysis of Various Metal-EC₂S₂Complexes.

This embodiment describes the extraction and purification of pine bark proanthocyanidins and synthesis of EC₂S₂ ligands (Compound 8), and preparation of a metal-EC₂S₂ complexes.

In a flask (50 mL), Pine Bark OPCs (2.8 g) was dissolved in 1% HCl Dioxane-water solution (v/v=1:1, 30 mL), 1,2-ethanedithiol (180 mL, 2.1 mmol) was then added. The mixture was kept at room temperature for 12 h with stirring. The reaction solution was dissolved in ethyl acetate (150 mL) and washed with 0.1M NaHCO₃. The organic fraction was dried over anhydrous sodium sulphate. Evaporation of the ethyl acetate gave dark brown residue, which was purified with column chromatography (silica gel, dichloromethane-methanol 8:1) to afford EC₂S₂ (100 mg, 7%) as a white solid. Analysis of the product revealed ¹H-NMR (300 MHz, CD₃OD): δ 7.01 (s, 1H), 6.84 (d, J=8.0 Hz, 1H), 6.78 (d, J=8.0 Hz, 1H), 5.97 (s, 1H), 5.92 (s, 1H), 5.29 (s, 1H), 4.05 (s, 1H), 3.35 (s, 3H), 2.96-3.18 (m, 2H) ppm. ¹³C NMR (75 MHz, CD₃OD): δ 159.1, 158.8, 157.2, 146.0, 145.8, 132.0, 119.4, 116.0, 115.3, 100.3, 96.8, 95.8, 75.6, 72.3, 43.7, 33.9 ppm. HRMS calcd for C₃₂H₂₉O₁₂³²S₂: 669.1106, found, 669.1107.

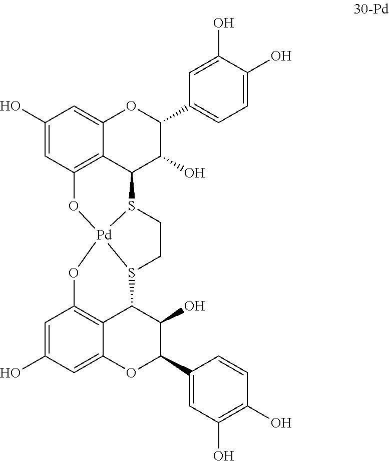

Compound 8 prepared from Pine Bark OPCs (4.91 mg) was dissolved in MeOH and 1 equivalent of Pd(II) acetate was dissolved in MeOH as well. They were then added in the presence of N₂ to prepare the Pd-Compound 8 complex (Compound 30-Pd). ¹H-NMR (300 MHz, CD₃OD): δ 7.39 (d, 2H), 7.23 (d, 1H), 6.65 (s, 1H), 6.54 (s, 1H), 5.10 (s, 1H), 4.28 (s, 1H), 3.35 (s, 3H), 2.77-2.99 (m, 2H) ppm. The ESI-MS (anionic mode) Compound 30-Pd (¹⁰⁶PdC₃₂H₂₇O₁₂³²S₂) complex at m/z 773.3, calcd 774.0.

Superoxide Dismutase Assay of Various Metal-EC₂S₂ Complexes.

EC₂S₂ synthesized from Pine Bark OPCs (400 μM) was mixed at room temperature in MeOH with 400 μM of Iron(II), Manganese(II), Nickel(II) and Copper(II) acetate salts respectively, in metal-ligand molar ratio of 1:1, followed by shaking for 10 seconds to prepare the four metal-EC₂S₂ complexes and their SOD activity was analyzed by using the SOD assay described below. The EC₂S₂ and the four metal acetates were also analyzed for SOD activity for comparisons.

Samples (20 μL of 400 μM) were manually pipetted into individual wells of a 96-well flat-bottom microplate in triplicates, followed by dispensing 1604 hydroethidine (HE) working solution (31.7 μM) prepared in pH 7.4 phosphate buffer into all the wells. The microplate was incubated at 37° C. for 10 minutes, before 20 μL xanthine oxidase (XO) (0.185 U/mL) prepared in pH 7.4 phosphate buffer was dispensed. The total liquid volume per well was 200 μL. Phosphate buffer control (20 μL) in presence and absence of XO were also run together in the same plate. The microplate was shaken for 10 seconds at an intensity of one. Fluorescence intensity was then recorded every 3 minutes for 20 minutes with a Synergy HT microplate fluorescence reader from Bio-Tek Instruments, Inc. The kinetic experiments were conducted by following the rate of oxidation of HE to E⁺ using excitation wavelength of 485 nm and emission wavelength of 645 nm. At saturating concentration of HE, dismutation of superoxide was considered to be negligible. In the absence of a SOD mimic, superoxide would be consumed by HE to generate the fluorescent product. However, when a SOD mimic is present, it would compete with HE for the superoxide and thus inhibit the oxidation of HE, resulting in a decrease in the rate of fluorescence produced.

To analyze the data, the rate of fluorescence produced in the phosphate buffer control in presence of XO was denoted as Vₒ, equivalent to 0% inhibition and this was related to the flux rate of superoxide. The rate of fluorescence produced in the phosphate buffer control in absence of XO was denoted as V_{blank}, equivalent to 100% inhibition. The rate of fluorescence produced in the tested samples was denoted as

V and the percentage of inhibition was calculated to determine their SOD activity. For detailed analytical method is documented in literature (Zhang, L., Huang, D.; Kondo, M.; Fan, E.; H.; Kou, Y.; Ou, B. Novel High-Throughput Assay for Antioxidant Capacity against Superoxide Anion, J. Agric. Food Chem. 2009, 57, 2661-2667.)

All the four Cu(II), Mn(II), Ni(II) and Fe(II)-EC₂S₂ complexes showed good SOD mimetic activity with percentage of inhibition being 90%, 77.8%, 76.3% and 72.5% respectively.

### Oxygen Radical Absorbance Assay

Peroxyl radical scavenging capacity of the EC₂S₂ was determined using oxygen radical absorbance capacity (ORAC) assay (Huang, D.; Ou, B.; Hampsch-Woodill, M.; Flanagan, J. A.; Prior, R. L. High-throughput assay of oxygen radical absorbance capacity (ORAC) using a multichannel liquid handling system coupled with a microplate fluorescence reader in 96-well format. Journal of Agricultural and Food Chemistry (2002), 50(16), 4437-4444.). The kinetic curves from the ORAC assay show dose dependent fashion with clear lag phase comparable to Trolox standard. The net area under the curve has excellent linear relationship with the concentration of EC₂S₂. The ORAC value calculated from the individual concentration is 10.79±0.58 μmol TE/μmol sample. This is the highest ORAC value reported for pure antioxidant compounds.

The invention provides an inexpensive alternative to costly synthetic chiral ligands for asymmetric reactions and replaces them with multi-dentate ligands easily derived from cheap and naturally occurring oligomeric proanthocyanidins (OPCs). Structurally, OPC has some similarity with (R or S)-BINAP, a “privileged” chiral ligand found many application in asymmetric organic reactions). Yet, optically active BINAP takes a few steps to synthesize, whereas, OPC is readily available from biomass.

The EC₂S₂ ligand synthesized from Pine Bark OPCs' antioxidant activity is two times higher than EC, indicating that it is a good antioxidant. The metal complexes of EC₂S₂ ligand show good SOD mimetic activity and may fulfil the role as synthetic low molecular weight SODs.

In conclusion, the Compound 8 with potential application as antioxidant has been obtained from plant materials. The new molecule can be easily separated from complex mixtures of plant materials by normal silica gel chromatography.

The ligand Compound 30 and their metal complexes (eg, compound 30-Pd, compound 30-Pt, etc) can be used in any catalytic reactions including but not limited to asymmetric hydrogenation, epoxidation, oxidation, reduction, substitution, addition, coupling, carbon carbon bond forming, carbon oxygen bond forming, carbon nitrogen bond forming reactions, kinetic resolution, carbon carbon double bond metathesis, and carbon carbon triple bond metathesis.

The Compound 8 and its metal complexes (FIG. 6) may serve as new therapeutic agents for cancer, for example, Compound 30-Pt complex). The Compound 30-Tc complex may be used for radiotherapy.

Having described the invention by reference to the foregoing embodiments, a skilled addressee will understand that further chiral ligands may be synthesised with alternative reagents, without departing from the spirit and scope of the invention.

1. A method for modifying a compound having repeat units of 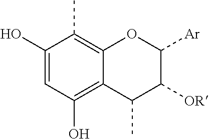
wherein Ar represents a substituted functional group selected from the group comprising a phenyl, a hydroxyphenyl, a dihydroxyphenyl, an alkoxy, an ester, an alkyl group, and an alkoxyphenyl group; the method comprising depolymerizing the compound with a nucleophile in the presence of acid.

2. A method as claimed in claim 1 wherein the nucleophile is selected from a group of compounds containing: sulphur, carbon, nitrogen, iodine, phosphorus, and arsenic atoms.

3. A method as claimed in claim 2 wherein the nucleophile is a compound containing carbon, and is selected from a group consisting of: heterocyclic compounds, aromatic compounds, acyclic organic compounds and small inorganic anions.

4. A method as claimed in claim 1 further comprising selectively protecting the hydroxyl groups in the repeat unit 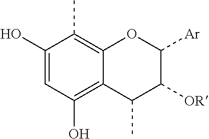
by adding a mixture of the compound having the repeat units in a polar aprotic solvent to 4-N,N-dimethylaminopyridine and methyl propiolate.

5. A method of synthesising a catechin from an oligomeric proanthocyanidin, comprising modifying selected polar oxygen containing groups with an unsaturated hydrocarbon or hydrocarbon derivative compound to prevent competitive binding of metals thereto, to form a modified oligomeric proanthocyanidin, and depolymerising the modified oligomeric proanthocyanidin to form said catechin in the form of a chiral ligand.

6. A method as claimed in claim 5 wherein the oligomeric proanthocyanidin is an oligomeric proanthocyanidin, and the intermediate oligomer is a modified oligomeric proanthocyanidin.

7. A method as claimed in claim 5 wherein the unsaturated hydrocarbon compound is selected from a group consisting of: a terminal alkyne and a terminal alkyne derivative.

8. A method as claimed in claim 5 wherein the modification of the oligomeric proanthocyanidin is by reaction with propynoate methyl ester (methyl propiolate).

9. A method as claimed in claim 5 wherein the modification of the oligomeric proanthocyanidin is by reaction in a polar aprotic solvent with propynoate methyl ester and N,N-dimethylpyridine.

10. A method as claimed in claim 5 wherein the oligomeric proanthocyanidin may conveniently have epicatechin as the monomeric unit.

11. A method as claimed in claim 10 wherein the selected polar oxygen containing groups comprise at least one hydroxyl group on the B ring of the epicatechin.

12. A method as claimed in claim 5 wherein in the step of depolymerizing, the modified oligomeric anthocyanidin is depolymerized with a nucleophile in the presence of an acid.

13. A method as claimed in claim 12 wherein the nucleophile is selected from a group of compounds containing: sulphur, carbon, nitrogen, iodine, phosphorus, and arsenic.

14. A method as claimed in claim 13 wherein the nucleophile is selected from a group of carbon nucleophiles containing: heterocyclic compounds, aromatic compounds, acyclic organic compounds and small inorganic anions.

15. A catechin metal complex comprising a catechin formed according to the method of claim 5 complexed with a metal selected from one or more of an alkali metal, and alkali earth metal, a transition metal, a lanthanide, an actinide, or a metalloid.

16. A compound having at least one unit of a general formula: 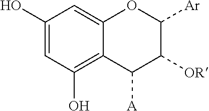
wherein Ar represents a substituted functional group selected from a group consisting of: a hydroxyphenyl, a dihydroxylphenyl, an alkoxyphenyl, ester, alkyl, alkoxyphenyl; and wherein A represents a substituted functional group.

17. A compound as claimed in claim 16 wherein A includes a nucleophile containing iodine, phosphorus, sulphur, oxygen, nitrogen, hydrogen, carbon, and any combination thereof.

18. A compound as claimed in claim 17 wherein the nucleophile A contains carbon, and the nucleophile is selected from a group consisting of: carbon-carbon single bonds, carbon-carbon double bonds, carbon-carbon triple bonds, nitrogen-carbon single bonds, nitrogen-carbon double bonds, sulphur-carbon single bond, oxygen-carbon single bond oxygen-carbon double bond, carbon-phosphine single bond, carbon iodine single bond, and any combination thereof.

19. A compound as claimed in claim 16 further including at least one metal selected from a group consisting of: alkali metals, alkali earth metals, transition metals, lanthanides, actinides, and metalloids.

20. A compound as claimed in claim 19 wherein the compound further includes a ligand bound to the metal, where the ligand is selected from a group consisting of: a monodentate, bidentate, tridentate, tetradentate, and pentadentate ligand.

21. A compound having at least one unit of a general formula: 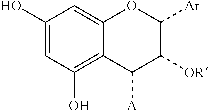
wherein Ar represents a substituted functional group selected from a group consisting of: a hydroxyphenyl, a dihydroxyphenyl, an alkoxyphenyl, ester, alkyl, alkoxyphenyl; where R′ is selected from hydrogen, any carbon containing moiety or other functional group; and
wherein A is selected from one of:
any moiety or moieties containing iodine or phosphorus;
a group having the formula —SCH₂CR_{A}R_{B}, where R_{A} is any functional group or moieties containing functional groups, and R_{B} is any group containing sulphur, or nitrogen and/or a cyclic, heterocyclic, polycyclic or polyheterocyclic moiety;
a group having the formula —S—CH₂CH—YR_{B}, where R_{B} is any group, and Y is sulphur, or nitrogen as a secondary or tertiary amine or an imine;
a group having the formula —S—R_{C}—YR_{B}, where Y is sulphur, or nitrogen as a secondary or tertiary amine or an imine, R_{B} is any group, and R_{C} is any group;
a group having the formula —R_{C}—YR_{B}, where Y is sulphur, or nitrogen as a secondary or tertiary amine or an imine, R_{B} is any group, and R_{C} is any group
a group selected from: 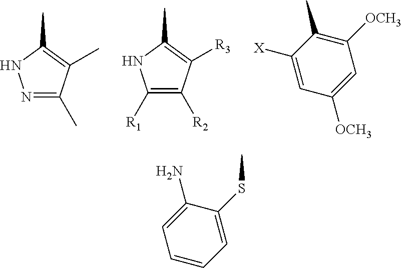
where X is selected from —OH and —NH2, and R1, R2, R3 are any group.

22. A compound as claimed in claim 21 wherein the group identified as Ar may comprise:

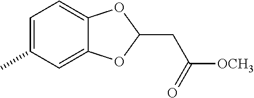

23. A compound as claimed in claim 21 wherein compound may, in a particularly advantageous embodiment, have the general formula:

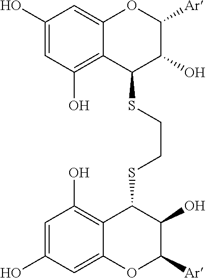

24. A compound as claimed in claim 21 complexed with a metal selected from the group consisting of: alkali metals, alkali earth metals, transition metals, lanthanides, actinides, and metalloids.

25. A compound as claimed in claim 24 further including at least one ligand bound to the metal.

26. A compound as claimed in claim 25 where the ligand is a multidentate ligand.

27. A compound as claimed in claim 24 further including a donor atom bound to the metal, the donor atom being selected from oxygen, nitrogen, sulphur, phosphorus, and carbon.

28. A compound having first and second units of a general formula: 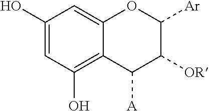
wherein Ar represents a substituted functional group selected from a group consisting of: a hydroxyphenyl, a dihydroxyphenyl, an alkoxyphenyl, ester, alkyl, alkoxyphenyl; where R′ is selected from hydrogen, any carbon containing moiety or other functional group; and
wherein A is selected from at least a moiety containing: iodine, phosphorus, sulphur, arsenic, carbon, nitrogen, oxygen and said first unit and said second unit are connected by A.
